(19) United States
(12) Patent Application Publication
(10) Pub. No.: US 2026/0263528 A1
(43) Pub. Date: Sep. 10, 2026

(54) Use of Hair Stem Cells in Wound Healing

(71) Applicant: CUTISS AG, Schlieren (CH)

(72) Inventors: Vincent RONFARD, Villarzel (CH); Paul KEMP, Llandudno (GB)

(21) Appl. No.: 19/489,268

(22) PCT Filed: Jul. 7, 2024
(86) PCT No.: PCT/IL2024/050660

§ 371 Date: Dec. 2, 2025

Related U.S. Application Data
(60) Provisional application No. 63/512,628, filed on Jul. 9, 2023.

(51) Int. Cl. A61K 35/36; A45D 26/00; A61F 2/10; A61K 47/44; A61P 17/02

(52) U.S. Cl. CPC A61K 35/36; A45D 26/0014; A61F 2/10; A61K 47/44; A61P 17/02

(57) ABSTRACT

A method comprising: (a) harvesting hair follicles by applying a wax to a first area of skin with hair follicles protruding therefrom; and (b) removing said wax from said first area and applying it to a wound in a second area of the skin so that said hair follicles and their keratinocyte stem cells contact said wound. Related compositions, articles of manufacture, and medical devices are also disclosed.

### CROSS REFERENCE TO RELATED APPLICATIONS

This PCT application claims the benefit according to 35 U.S.C. § 119(e) of U.S. provisional patent application 63/512,628 filed on 9/Jul/2023 and having the same title and inventors as the present application; which is fully incorporated herein by reference.

### FIELD OF THE INVENTION

The invention is in the field of stem-cell based grafts or transplants.

### BACKGROUND OF THE INVENTION

Wound healing is a major problem in modern medicine.

More than 13 million people suffer worldwide from chronic wounds per year. Many factors can lead to impaired healing. The primary factors are hypoxia, bacterial colonization, ischemia, reperfusion injury, altered cellular response, and collagen synthesis defects.

Aberrant wound healing is rarely seen in normal healthy subjects. Aberrant wound healing is usually associated with comorbidities (e.g., diabetes, autoimmune disease, peripheral artery disease), increased body mass index, anatomic location, and medications.

A chronic wound may be defined as a common type of skin injury that is physiologically impaired due to a disruption of the wound healing cycle and more specifically because this healing process stops at the inflammatory phase. A wound is considered as chronic when it has not healed within six weeks and shows signs of prolonged inflammation, a defective wound matrix and failure of re-epithelialization. In this case, the duration of wound healing varies with the wound's type and severity and some never happen completely. Local treatment is applied to reduce pain and itching, infection and bleeding from the wound, and tries to deal with excess exudate that can lead to unpleasant odors, element that is very troublesome for the patients.

Chronic non-healing wounds such as diabetic foot ulcers, venous leg ulcers, and pressure ulcers present special challenges.

Acute skin wounds such as burns present a separate, but no less difficult, set of challenges.

Wound healing is a natural physiological reaction to tissue injury typically lasting 4 to 6 weeks. The healing process can be divided into four steps: hemostasis, inflammation, proliferation, and remodeling.

The healing process involves numerous cell types, cytokines, mediators, and the vascular system. A cascade of initial vasoconstriction of blood vessels and platelet aggregation is designed to stop bleeding. This cascade is followed by an influx of a variety of inflammatory cells, starting with the neutrophils. These inflammatory cells, in turn, release a variety of mediators and cytokines to promote angiogenesis, thrombosis, and re-epithelialization. The fibroblasts, in turn, lay down extracellular components which will serve as scaffolding.

During several days, the inflammatory phase is put in place for limiting further damage, closing the wound, removing cellular debris and bacteria, and fostering cellular migration. This step is characterized by hemostasis, chemotaxis, and increased vascular permeability. The mediators, cytokines and other factors secreted by neutrophils, monocytes, endothelial cells and platelets play a role in promoting collagen degradation, the transformation of fibroblasts, the growth of new vessels, cellular permeability and re-epithelialization.

At the same time, the proliferative phase started with the formation of granulation tissue, re-epithelialization, and neovascularization.

Production of collagen by fibroblasts happens with the migration of cells from the wound from the wound periphery and adjacent edges which allows the re-epithelialization. The neovascularization, angiogenesis and vasculogenesis occur by forming new blood vessels from existing vessels.

The remodeling phase is the phase where the excess collagen degrades, and wound contraction happens, so the wound achieves the maximum strength that represents around 80% of the tensile strength of normal skin.

U.S. Pat. No. 7,419,661 describes the use of dermal sheath tissue and/or cells derived therefrom in wound healing systems. The relevant features of this tissue type are exploited to provide a new wound healing material that purports to have application in the provision of new therapeutic compositions and new surgical dressings where wound closure and minimal scarring is desirable.

WO2011/104030 describes a method for promoting the healing of superficial wounds or the restoration of the function of the skin and/or the skin appendages thereof of a second skin region, having the following steps: applying cells that were obtained from hair root sheaths of a first skin region of a donor onto a second skin region of a recipient. The application further relates to cells, to a preparation, to the use of cells, and to a method for producing a preparation.

WO2011050948 describes a tape, in particular adhesive tape, for the treatment of skin disorders comprising at least one base layer having a first surface facing away from the skin during use and a second surface facing towards the skin during use, and at least one first film, in particular dissolvable film, on the side of said second surface, which covers at least part of the second surface of said base layer, said first film, in particular dissolvable film, having a first surface facing away from the skin during use and a second surface facing towards the skin during use and comprising at least one enzyme, in particular a proteolytic enzyme.

EP2343079B1 describes a method and or device suitable for producing a transplantable cellular suspension of living tissue suitable for grafting to a patient. In applying the method and or in using the device donor tissue is harvested, subjected to a cell dissociation treatment, cells suitable for grafting back to a patient are collected and dispersed in a solution that is suitable for immediate dispersion over the recipient graft site.

WO2016131096A1 describes methods and products for delivering cells to a biological site. The application purports to provide a method of delivering cells to a biological site including providing a product comprising an alkylamine functionalised substrate and cells for delivery to the biological site attached to the functionalised substrate, wherein the alkylamine functionalised substrate comprises a surface density with an atomic ratio of primary amine to carbon of greater than 0.005, and applying the product to the biological site to allow transfer of the cells from the product to the biological site, thereby delivering cells to the site.

EP1198557B1 describes the treatment of skin defects by organotypically-cultured autologous keratinocytes isolated from the outer root sheath of anagen or growing hair. Methods for primary, as well as subsequent organotypic cultures (i.e., epidermal equivalents) in fully-defined media supplemented by autologous human serum and substances isolated form blood components, with minimal allogeneic biological supplements, are disclosed herein.

### SUMMARY OF THE INVENTION

A broad aspect of some embodiments of the invention relates to application of cells to a wound without prior cultivation or expansion.

One aspect of some embodiments of the invention relates to the use of wax as a transfer medium for keratinocyte stem cells without culture or expansion of the cells. In some embodiments elimination of cell culture/expansion contributes to a reduction in treatment duration. Alternatively or additionally, use of wax as a transfer medium contributes to a reduction in invasiveness, and/or obviates a need for surgery. In some exemplary embodiments of the invention, the use of wax as a transfer medium contributes to a reduction in a patient's pain and/or promotes faster wound healing and/or allows the patient to resume normal daily activities more quickly.

Another aspect of some embodiments of the invention relates to grafting of keratinocyte stem cells located at the tip of plucked hairs of a donor to an open wound without culturing the keratinocyte stem cells. In some exemplary embodiments of the invention, wax is used as a transfer vehicle. In some exemplary embodiments of the invention, the grafting is autologous grafting. In some exemplary embodiments of the invention, before grafting onto the wound, necrotic debris or nonviable tissue is/are removed. Alternatively or additionally, in some embodiments the wound bed to is prepared to accept a skin graft or flap, or for closure, when indicated.

Another aspect of some embodiments of the invention relates to use of wax as a transfer vehicle to harvest hair follicles at a first location and transfer them to a wound bed at a second location. According to various exemplary embodiments of the invention the wax is applied at the first location as a liquid (melted), a paste, or as a solid (sheet or strip) to remove hair from normal skin. The wax is then removed and applied to a wound so that keratinocyte stem cells located at the tip of plucked hairs contact the wound bed. In some embodiments the wax is applied using a dressing which covers the wax while holding it in place on the wound bed. According to various exemplary embodiments of the invention the first location includes legs and/or neck and/or arms. Alternatively or additionally, according to various exemplary embodiments of the invention the hair is applied to a second location on the subject that donated the hair (autologous graft).

According to various exemplary embodiments of the invention the wax is impregnated with growth factors and/or antibiotics and/or cytokines and/or extracellular matrix molecules. In some exemplary embodiments of the invention, hair follicles harvested on a wax surface are treated with one or more biologically active agents before being applied to the wound. According to various exemplary embodiments of the invention this treatment includes, for example, dipping in or spraying with a solution of the biologically active agent(s). According to various exemplary embodiments of the invention the biologically active agents include antimicrobial compounds such as silver, betaine, chitin, polyhexamethylene biguanide, antibiotics and/or anti-fungal substances.

Alternatively or additionally, in some embodiments the biologically active agents include epidermal growth factor (EGF) and/or fibroblast growth factor (FGF) and/or transforming growth factor-β (TGF-β) and/or platelet derived growth factor (PDGF), and or vascular endothelial growth factor (VEGF)m and/or interleukins such as IL1 and/or IL 6) and/or tumor necrosis factor-α (TNF-α). In some exemplary embodiments of the invention, one or more of the factors contribute to a decrease in perturbations in growth factors and cytokines occur in a chronic wound.

According to various exemplary embodiments of the invention the treatment enhances cell growth and/or differentiation and/or removes debris and/or inactivates unwanted microorganisms.

For purposes of this specification and the accompanying claims, the term “wax” is to be construed broadly so that it includes not only long-chain fatty acids linked through an ester oxygen to a long-chain alcohol but also other polymers such as thermosensitive polymers and/or UV sensitive polymers and/or carbohydrate polymers or other materials sufficiently adhesive to allow hair removal from normal skin areas and which is biocompatible for application to chronic wounds.

Although the aspects are presented separately hereinabove, according to various exemplary embodiments of the invention two, or all three, of the aspects and/or their features are combined to create additional embodiments of the invention.

It will be appreciated that the various aspects described above relate to solution of technical problems associated with reducing the invasive nature of stem cell harvesting.

Alternatively or additionally, it will be appreciated that the various aspects described above relate to solution of technical problems related to reducing the amount of time needed to prepare an autologous graft.

Alternatively or additionally, it will be appreciated that the various aspects described above relate to solution of technical problems associated with treatment of dermatological damage such as chronic wounds including but not limited to nonhealing or infected surgical or traumatic wounds, venous ulcers, arterial ulcers, pressure ulcers (e.g. bedsores, decubitus ulcer, or pressure sores), diabetic foot ulcers, and ischemic ulcers, Epidermolysis bullosa, Pyoderma gangrenosum, wounds after excision of giant congenital nevi, and acute wounds, in humans and/or animals.

Alternatively or additionally, it will be appreciated that the various aspects described above relate to solution of technical problems associated with post-operative treatment following the removal of tattoos, naevi, skin cancer, papillomas, after amputation, or rejuvenation of actinically damaged skin after skin resurfacing.

Alternatively or additionally, it will be appreciated that the various aspects described above relate to solution of technical problems associated with amelioration of scars by providing Keratinocyte stem cells to the wound.

In some exemplary embodiments of the invention there is provided a method comprising: (a) harvesting hair follicles containing keratinocyte stem cells by applying wax to a first area of skin with hair follicles protruding therefrom; and (b) removing the wax from the first area and applying it to a wound in a second area of the skin so that the hair follicles and their keratinocyte stem cells contact the wound. In some embodiments the method includes priming the hair follicles containing keratinocyte stem cells prior to the applying to the wound. Alternatively or additionally, in some embodiments the method includes washing the wax and/or the hair follicles prior to the applying to the wound. Alternatively or additionally, in some embodiments the method includes treating the wax and/or the hair follicles containing the keratinocyte stem cells with one or more antibiotics prior to the applying to the wound.

In some exemplary embodiments of the invention there is provided a wax composition with hair follicles containing keratinocyte stem cells adhered thereto for use in a method of wound treatment comprising application of the composition to a wound so that the hair follicles containing the keratinocyte stem contact the wound. In some embodiments the composition for use includes hair follicles containing keratinocyte stem cells which remain adhered to the composition during the application. Alternatively or additionally, in some embodiments, the method includes priming the hair follicles containing keratinocyte stem cells prior to the applying to the wound. Alternatively or additionally, in some embodiments, the method includes washing the wax and/or the hair follicles containing keratinocyte stem cells prior to the applying to the wound. Alternatively or additionally, in some embodiments the method includes treating the wax and/or the hair follicles containing keratinocyte stem cells with one or more antibiotics prior to the applying to the wound.

In some exemplary embodiments of the invention there is provided a composition comprising wax impregnated with at least one member of the group consisting of antibiotics, growth factors, cytokines and extracellular matrix molecules.

In some exemplary embodiments of the invention there is provided an article of manufacture comprising a depilatory wax strip or sheet with through perforations distributed on a surface thereof which contacts skin during use.

In some exemplary embodiments of the invention there is provided a medical device comprising a wax composition with hair follicles containing keratinocyte stem cells adhered thereto for treating dermatological damages. In some embodiments, the dermatological damages are selected from the group consisting of nonhealing or infected surgical or traumatic wounds, venous ulcers, arterial ulcers, pressure ulcers such as bedsore, decubitus ulcer, or pressure sore, diabetic foot ulcers, ischemic ulcers, Epidermolysis bullosa, Pyoderma gangrenosum, and wounds after excision of giant congenital nevi, in humans and animals. Alternatively or additionally, in some embodiments, the dermatological damages are chosen in the group consisting of treatment of post-operative damage following the removal of tattoos, naevi, skin cancer, papilloma, after amputation, rejuvenation of actinically damaged skin after skin resurfacing.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office. Thus, any recitation that an embodiment “includes” or “comprises” a feature is a specific statement that sub embodiments “consist essentially of” and/or “consist of” the recited feature.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The phrase “adapted to” as used in this specification and the accompanying claims imposes additional structural limitations on a previously recited component.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Percentages (%) of chemicals are W/W (weight per weight) unless otherwise indicated.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a simplified flow diagram of a method according to some embodiments of the invention; and

FIG. 2 is a schematic representation of an article of manufacture according to some embodiments of the invention.

### DETAILED DESCRIPTION OF EMBODIMENTS

Embodiments of the invention relate to methods and related compositions and article of manufacture for harvest of hair follicle containing keratinocyte stem cells and application of those keratinocyte stem cells to a wound.

Specifically, some embodiments of the invention can be used to promote wound healing by grafting uncultured keratinocyte stem cells to a wound.

The principles and operation of methods, compositions and articles of manufacture according to exemplary embodiments of the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

### Exemplary Method

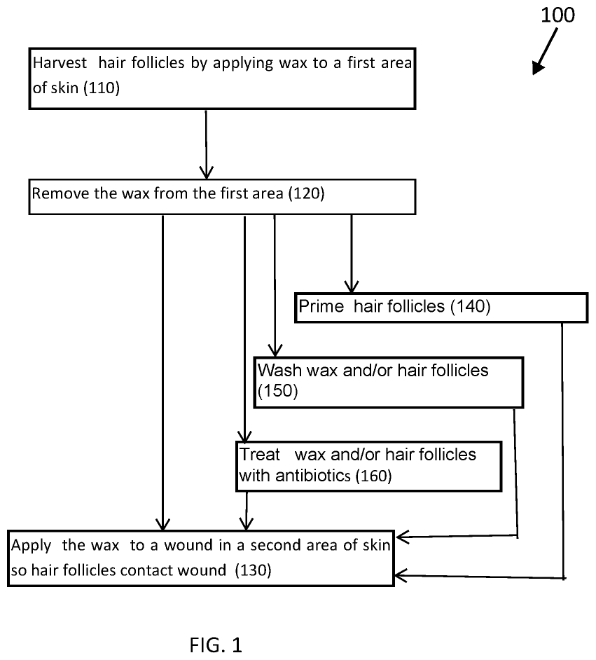

FIG. 1 is a simplified flow diagram of a method, indicated generally as 100, for preparing a skin graft in situ according to some embodiments of the invention.

Depicted exemplary method includes harvesting 110 keratinocyte stem cells by applying wax to a first area of skin with hair follicles protruding therefrom and removing 120 the wax from the first area and applying 130 it to a wound in a second area of the skin so that the hair follicles adhering to the wax and their keratinocyte stem cells contact the wound bed. According to these embodiments, the wax is left on the wound for days or weeks so that the keratinocyte stem cells grow and populate the wound. The expanded population of keratinocyte stem cells serves as a skin graft which covers the wound.

In some embodiments method 100 includes priming 140 the keratinocyte stem cells located on the hair follicles prior to applying 130. Priming 140 prepares the cells to be more effective for healing. In some exemplary embodiments of the invention, priming 140 is done 3, 4, 5, 7, 14, 21, 28 or more or intermediate numbers of days in advance. One method of priming involves inducing cell proliferation by rubbing the skin every day for a certain period of time. According to various exemplary embodiments of the invention the period of time is 5, 10, 15, 20, 25 or 30 minutes or intermediate or longer times at a frequency of 1, 2, 3 or more times per day.

Alternatively or additionally, in some embodiments method 100 includes preparation of the wound prior to applying 130. According to various exemplary embodiments of the invention reparation of the wound includes cleaning and/or formation of granulation tissue and/or removal of fibrinous tissue.

Alternatively or additionally, in some embodiments method 100 includes washing 150 the wax and/or the hair follicles containing the keratinocyte stem cells prior to applying 130.

Alternatively or additionally, in some embodiments method 100 includes treating 160 the wax and/or the hair follicles containing the keratinocyte stem cells with one or more antibiotics and/or growth factors and/or cytokines and/or extracellular matrix molecules prior to applying 130.

In some exemplary embodiments of the invention, hair follicles harvested on a wax surface are treated with one or more biologically active agents before being applied to the wound. According to various exemplary embodiments of the invention this treatment includes, for example, dipping in or spraying with a solution of the biologically active agent(s). According to various exemplary embodiments of the invention the biologically active agents include antimicrobial compounds such as silver, betaine, chitin, polyhexamethylene biguanide, antibiotics and/or anti-fungal substances.

Alternatively or additionally, in some embodiments the biologically active agents include epidermal growth factor (EGF) and/or fibroblast growth factor (FGF) and/or transforming growth factor-β (TGF-β) and/or platelet derived growth factor (PDGF), and or vascular endothelial growth factor (VEGF)m and/or interleukins such as IL1 and/or IL 6) and/or tumor necrosis factor-α (TNF-α). In some exemplary embodiments of the invention, one or more of the factors contribute to a decrease in perturbations in growth factors and cytokines occur in a chronic wound.

In many exemplary embodiments of method 100, the first area of skin from which the hair follicles containing the keratinocyte stem cells are harvested and the second area of skin where the wound is located, are on a body of a same person. According to these embodiments, method 100 and 200 is a method of autologous skin grafting.

### Exemplary Composition for Use

In some exemplary embodiments of the invention there is provided a wax composition for use in a method of wound treatment which includes harvesting hair follicles containing keratinocyte stem cells by adhering the follicles to the composition; and application of the harvested hair follicles containing the keratinocyte stem cells to a wound. The method itself is described hereinabove and diagrammed in FIG. 1.

In some embodiments of the composition for use, the hair follicles containing the keratinocyte stem cells remain adhered to the composition during application to the wound.

Alternatively or additionally, in some embodiments of the composition for use, the method includes priming the hair follicles containing the keratinocyte stem cells prior to applying them to the wound. Priming is as described above. Alternatively or additionally, in some embodiments of the composition for use, the method includes washing the wax and/or the hair follicles containing the keratinocyte stem cells prior to applying to the wound. Alternatively or additionally, in some embodiments of the composition for use, the method includes treating the wax and/or the hair follicles containing the keratinocyte stem cells with one or more antibiotics prior to applying to the wound.

In some exemplary embodiments of the invention there is provided a composition comprising wax impregnated with at least one member of the group consisting of antibiotics, growth factors, extracellular matrix components, autologous growth factors/molecules such as PRP, Fibrinogen, cytokines, extracellular matrix molecules, silver, betaine, chitin, polyhexamethylene biguanide, anti-fungal substances, epidermal growth factor (EGF), fibroblast growth factor (FGF), transforming growth factor-β (TGF-β), platelet derived growth factor (PDGF), vascular endothelial growth factor (VEGF) and/or interleukins (e.g. IL1 and/or IL 6), tumor necrosis factor-α (TNF-α).

Extracellular matrix components include, but are not limited to collagen, fibrin, laminin, GAGs, and HA. In some exemplary embodiments of the invention, the composition is provided as preformed strips or sheets. In some embodiments these units are provided as a continuous roll with perforation between the units to facilitate separation of one unit from another. In other exemplary embodiments of the invention, the wax is provided as a block to be melted prior to application.

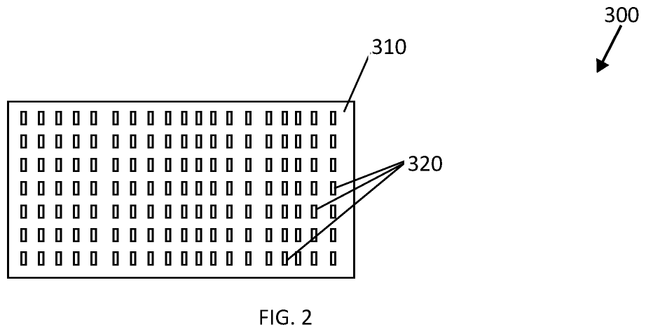

FIG. 2 is a schematic representation of an article of manufacture, indicated generally as 300 according to some embodiments of the invention. In the depicted embodiment, the article of manufacture includes a depilatory wax strip or sheet with through perforations 320 distributed on a surface 310 thereof which contacts skin during use. Such a strip or sheet is useful in practice of methods 100 and/or 200 as described hereinabove. Perforations 320 contribute to air permeability and/or moisture permeability of the strip/sheet 300.

### Exemplary Advantages

In some exemplary embodiments of the invention, a need for culturing keratinocyte stem cells is obviated.

Alternatively or additionally, in some embodiments a need to provide fibroblast stem cells or mesenchymal stem cells (MSCs) in order to produce a skin graft is obviated.

Alternatively or additionally, harvesting keratinocyte stem cells by plucking hair is a relatively non-invasive way of harvesting stem cells. Alternatively or additionally, hair regenerates quickly and automatically, so repeat harvesting over the course of time is available if needed.

### Exemplary Wax Properties

In some exemplary embodiments of the invention, the wax is biocompatible. For purposes of this specification and the accompanying claims, the term “biocompatible” means not harmful for the Human body and Human cells.

Alternatively or additionally, in some embodiments the wax is biodegradable or degrades at a predetermined rate. According to these embodiments, the wax will not need to be removed from the wound. In some embodiments, it takes about a week for the cells/tissue to engraft and a progressive degradation of the wax occurs during this time and/or beyond this time.

Alternatively or additionally, in some embodiments the wax is permeable to air and/or water. Permeability can be achieved, for example, by providing small perforations in a wax sheet or strip. According to these embodiments increasing a size or density (number per unit area), of perforations contributes to an increase in permeability. In some embodiments permeability contributes to an increase in wound healing rate by allowing CO₂ and water vapor released from cell respiration in the healing wound to escape to the environment.

### Exemplary Wax Compositions

According to various exemplary embodiments of the invention different wax compositions are employed.

In some embodiments, the wax comprises rosin (including resin acids) and/or modified rosin and/or beeswax and/or paraffin and/or an elastomer and/or other thermoplastic materials.

In other embodiments, the wax is a low temperature self-supporting wax, such as those known to those of ordinary skill in the art from EP 029 816. Low temperature waxes are heated to change from a solid consistency to a paste or fluid consistency.

In some exemplary embodiments of the invention, sugar waxes are employed. Sugar waxes comprise polysaccharides or monosaccharides (sucrose, glucose, fructose, dextrose, maltose, honey, maple syrup, invert sugars, or combinations thereof), water and citric acid. In some embodiments the sugar waxes are caramelized.

One exemplary wax composition includes 60 to 85% by weight of rosin type wax, 25 to 40% by weight of aqueous solution and 0.2 to 3% by weight of surfactant or a mixture of surfactants. In some exemplary embodiments of the invention, the rosin type wax includes a mixture of water and starch or cellulose pulp. In other exemplary embodiments of the invention, the rosin type wax includes a mixture of modified rosin, liquid paraffin, beeswax, microcrystalline wax. Alternatively or additionally, in some embodiments a wax based on natural pine resin is employed.

Alternatively or additionally, in some embodiments the wax composition further comprises from 0 to 10% by weight of one or more additives selected from the group consisting of perfumes, essential oils, opacifiers, antiseptics, analgesics, antibiotics, dyes, plasticizers, salts, emollients, coconut oil, and honey.

Alternatively or additionally, in some embodiments the wax contains one or more surfactants. Surfactants are amphiphilic substances having an affinity for the lipophilic phases and at the same time an affinity for the hydrophilic phases of an emulsion, which allows them to make the two phases compatible by being arranged around the dispersed globules of an emulsion. Thus, the addition of one or more surfactants, in particular of the water-in-oil type, in the appropriate proportions advantageously allows the production of stable mixtures. In some exemplary embodiments of the invention, glucolipid emulsifiers of plant origin are employed.

### Exemplary Dressings

In some exemplary embodiments of the invention, the wax is protected by a dressing. In some embodiments the dressing includes a low adherent dressing (e.g. gauze) and/or includes a semipermeable film (e.g. TEGADERM™; Chlorhexidine Gluconate).

Alternatively or additionally, in some embodiments the dressing includes hydrocolloids and/or hydrogels. Hydrocolloids and/or hydrogels contribute to an ability of the dressing to absorb exudate and/or contribute to an ability of the dressing to keep a moist environment. Hydrocolloids and/or hydrogels include but are not limited to alginates, seaweed-derived non-woven fibers, and collagen. Seaweed-derived non-woven fibers and collagen are suitable for use in highly exudative wounds because of their ability to absorb large amounts of fluid.

### Exemplary Use Scenarios

Various exemplary embodiments of the invention are used to treat chronic wounds including but not limited to non-healing or infected surgical or traumatic wounds, venous ulcers, arterial ulcers, pressure ulcers (e.g. bedsores, decubitus ulcer, or pressure sores), diabetic foot ulcers, and ischemic ulcers, Epidermolysis bullosa, Pyoderma gangrenosum, wounds after excision of giant congenital nevi, and acute wounds, in humans and/or animals.

Alternatively or additionally, various exemplary embodiments of the invention are used post-operatively following the removal of tattoos, naevi, skin cancer, papillomas, after amputation, or rejuvenation of actinically damaged skin after skin resurfacing.

Alternatively or additionally, various exemplary embodiments of the invention contribute to amelioration of scars by providing Keratinocyte stem cells to the wound.

In general, application of this technology is appropriate in any instance where epithelial coverage of a wound is needed.

Alternatively or additionally, in some embodiments the graft/transplant provides pigmentation. In some embodiments, melanocytes included in the hair follicle migrate and repopulate a depigmented area. In some embodiments the depigmented area is prepared by de-epithelialization to allow melanocytes to cover the wound with a pigmented surface layer.

It is expected that during the life of this patent many new wax formulations will be developed and the scope of the invention is intended to include all such new technologies a priori.

As used herein the term “about” refers to ±10 %.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Specifically, a variety of numerical indicators have been utilized. It should be understood that these numerical indicators could vary even further based upon a variety of engineering principles, materials, intended use and designs incorporated into the various embodiments of the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize an apparatus and features used to describe an apparatus can be used to characterize a method.

It should be further understood that the individual features and/or aspects described hereinabove are combined in all possible combinations and sub-combinations to produce additional embodiments of the invention. The examples given above are exemplary in nature and do not limit the scope of the invention which is defined solely by the following claims.

Each recitation of an embodiment of the invention that includes a specific feature, part, component, module or process is an explicit statement that additional embodiments of the invention not including the recited feature, part, component, module or process exist.

Alternatively or additionally, various exemplary embodiments of the invention exclude any specific feature, part, component, module, process or element which is not specifically disclosed herein.

Specifically, the invention has been described in the context of autologous grafts.

All publications, references, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “include”, and “have” and their conjugates as used herein mean “including but not necessarily limited to”.

Additional objects, advantages, and novel features of various embodiments of the invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

### Example 1

### Heterologous Mouse to Mouse Grafting

In order to provide proof of concept, hair follicles from the back of a normal (hairy) mouse were harvested using a commercially available waxing formula marketed to women for depilation. The wax formula contains rosin wax, beeswax, paraffin and film-forming agents, coconut oil and perfume. The wax containing the harvested hair follicles was grafted onto an open wound made on the back of nude mouse. For comparison, the wax without any hair was also grafted onto a wound on the back of other mice. Three weeks after grafting, the healed wound was harvested and neo epidermis made from migration of stem cells from the grafted epidermis was identified by histologic analysis of the tissue.

This example illustrates that freshly harvested hair follicles can spontaneously expand to form a graft without prior expansion of cell numbers in laboratory culture. Nude mice were employed to eliminate the potential for any immune reaction. Elimination of a potential immune reaction allowed examination of the ability of the allogenic grafted cells to “take” on the wound and contribute to the healing process. A moderate presence of exudates was observed on the compress of mice in both experimental groups between day 3 and day 7. The presence of exudates then decreased progressively in mice grafted with wax containing hair follicles compared to the other group for which it increased progressively until day 14 before decreasing progressively until day 28. No signs of infection/maceration were observed in mice grafted with wax containing hair throughout the experiment. Concerning mice grafted with wax alone, signs of infection/maceration were observed between day 7 and day 21 with a maximum on day 14. With the wax with hair follicles, obtained less exudates and less infection were observed.

### Example 2

### Human Xenograft to Mouse Grafting

In order to confirm the results of Example 1, hair follicles from a human subject were harvested using a commercially available waxing formula as in Example 1. Again, the wax containing the harvested hair follicles was grafted onto an open wound made on the back of a nude mouse. Three weeks after grafting, the healed wound was harvested and neo epidermis made from migration of stem cells from the grafted epidermis was identified by Histology analysis of the tissue.

This example confirms the results of Example 1 and suggests that cross species grafting using the described methods is feasible.

1. A method comprising:
(a) harvesting hair follicles by applying a wax to a first area of skin with hair follicles protruding therefrom; and
(b) removing said wax from said first area and applying it to a wound in a second area of the skin, without prior cultivation or expansion of said hair follicles, so that said hair follicles and their keratinocyte stem cells contact said wound.

2. A method according to claim 1, comprising priming said hair follicles containing keratinocyte stem cells prior to said applying to said wound.

3. A method according to claim, comprising washing said wax and/or hair follicles containing keratinocyte stem cells prior to said applying to said wound.

4. A method according claim 1, comprising treating said wax and/or hair follicles containing keratinocyte stem cells with one or more antibiotics prior to said applying to said wound.

5-11. (canceled)

12. A medical device comprising a wax composition with hair follicles containing keratinocyte stem cells adhered thereto for treating dermatological damages, wherein said keratinocyte stem cells adhered thereto are not subject to prior cultivation or expansion.

13. A medical device according to claim 12, characterized in that the dermatological damages are selected from the group consisting of nonhealing or infected surgical or traumatic wounds, venous ulcers, arterial ulcers, pressure ulcers such as bedsore, decubitus ulcer, or pressure sore, diabetic foot ulcers, ischemic ulcers, Epidermolysis bullosa, Pyoderma gangrenosum, wounds after excision of giant congenital nevi, a in humans and animals.

14. A medical device according to claim 12, characterized in that the dermatological damages are chosen in the group consisting of treatment of post-operative damage following the removal of tattoos, naevi, skin cancer, papilloma, after amputation, rejuvenation of actinically damaged skin after skin resurfacing.
